(19) United States
(12) Patent Application Publication
(10) Pub. No.: US 2007/0190171 A1
(43) Pub. Date: Aug. 16, 2007

(54) Methods for Altering Stool Quality and/or Stool Frequency

(76) Inventors: Ryan Michael Yamka, Topeka, KS (US); Kim Gene Friesen, Topeka, KS (US); Thomas Gordon Forster, Topeka, KS (US); Lauren Jay Kats, Topeka, KS (US)

Correspondence Address: COLGATE-PALMOLIVE COMPANY, 909 RIVER ROAD, PISCATAWAY, NJ, 08855, US

(21) Appl. No.: 11/566,512

(22) Filed: Dec. 4, 2006

Related U.S. Application Data
(60) Provisional application No. 60/741,632, filed on Dec. 2, 2005.

(51) Int. Cl. A61K 33/42; A61K 33/06

(52) U.S. Cl. 424/602; 424/682; 424/722

(57) ABSTRACT

Methods for altering and therefore improving stool quality and/or stool frequency for an animal by adjusting the balance of metabolizable cations to metabolizable anions consumed by the animal.

### CROSS-REFERENCE TO RELATED APPLICATION

This application claims priority to U.S. Provisional Application Ser. No. 60/741,632 filed Dec. 2, 2005, the disclosure of which is incorporated herein by reference.

### BACKGROUND OF THE INVENTION

1. Field of the Invention

The present invention relates generally to methods for altering stool quality and/or stool frequency for an animal and particularly to methods for altering stool quality and/or stool frequency for an animal by adjusting the balance of metabolizable cations to metabolizable anions consumed by the animal.

2. Description of the Related Art

Stool quality and stool frequency are generally determined by five factors: food ingredient digestibility, fiber level, health status, activity level, and water intake. When these factors are balanced, stools are generally formed, firm, dark, and exhibit a relatively reduced odor. Stools exhibiting these properties are considered to be good quality stools. If the factors are not balanced, stools are generally soft, loose, watery, light-colored, and exhibit a relatively increased odor. Stools exhibiting these properties, particularly loose, watery stools, are considered to be poor quality stools.

Poor stool quality and irregular stool frequency can be caused by various factors, e.g., abnormal intestinal motility, increases in intestinal permeability, the presence of nonabsorbable osmotically active substances in the intestine, or agents that cause diarrhea. Similarly, some animal foods, particularly those known in the art as chunk and gravy animal foods, can cause poor stool quality. Often, an animal consuming such foods has a fecal discharge that is irregular and undesirable. Such discharge is generally characterized by frequent loose, watery stools. In some instances, the discharge may be classified as diarrhea.

Methods for managing stool quality and stool frequency have focused on the use of drugs that combat diarrhea and on compositions that affect stool quality. For example, U.S. Pat. No. 6,280,779 discusses foods containing chemically modified starches and gums useful for maintaining normal bowel health and promoting production of acceptable stool quality. WO05063271A1 discusses the use of herbal compositions to treat gastro-intestinal disorders and affect stool quality. U.S. Pat. No. 5,919,760 discusses the use of octreotide to treat diarrhea; and WO9625940A1 discloses the use of compositions comprising carrots, rice, bananas and glucose for treating diarrhea. While these methods are useful, there continues to be a need for new methods and compositions for altering and therefore improving stool quality and stool frequency.

### SUMMARY OF THE INVENTION

It is, therefore, an object of the present invention to provide methods for improving stool quality for an animal.

It is another object of the invention to provide methods for altering stool frequency for an animal.

It is another object of the invention to provide articles of manufacture in the form of kits suitable for altering and improving stool quality and/or stool frequency.

It is a further object of this invention to provide means for communicating information about the methods and kits of this invention and their use for altering and improving stool quality and/or stool frequency.

These and other objects are achieved by adjusting the balance (ratio) of metabolizable cations to metabolizable anions consumed by an animal by an amount sufficient to alter and therefore improve stool quality and/or stool frequency for an animal. Kits comprising combinations of cations, anions, foods, compounds, instructions, and devices useful for altering stool quality and/or stool frequency are also provided.

Additional objects, features, and advantages of this invention will be apparent to those skilled in the art from reading this patent.

### DETAILED DESCRIPTION OF THE INVENTION

In one aspect, the present invention provides methods for improving stool quality for an animal. In another, the invention provides methods for altering stool frequency for an animal. The methods comprise adjusting the balance of metabolizable cations to metabolizable anions consumed by the animal by an amount sufficient to alter the animal's stool quality and/or stool frequency.

The term “animal” means any animal susceptible to or suffering from poor stool quality and/or irregular stool frequency. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

The methods are useful for a variety of human and non-human animals, including avian, bovine, canine, equine, feline, hicrine, murine, ovine, and porcine animals, and are particularly useful for companion animals such as canines and felines, including dogs and cats. In some embodiments, the animal is a member of the order Carnivora. In some such embodiments, the animal is a canine, and in other such embodiments a feline. In some embodiments, the animal is a companion anminal. A companion animal can be, for example, an animal of any species that is kept as a pet. A companion animal can also be an animal from a variety of widely domesticated species, for example, dogs (Canis familiaris) and cats (Felis domesticus) regardless of whether or not the animal is kept solely as a pet. Thus, companion animals include, for example, working dogs, cats kept for rodent control, as well as pet cats and dogs.

The cations and anions useful in the invention are any cation or anion suitable for consumption by an animal. In one embodiment, the metabolizable cations are selected from the group consisting of calcium, sodium, potassium, and magnesium and the metabolizable anions are selected from the group consisting of phosphorus, chloride, and sulfur.

The balance of metabolizable cations to metabolizable anions can be determined by any means known to skilled artisans. For example, one method for measuring the balance of metabolizable cations to metabolizable anions is to calculate the animal's dietary cation-anion balance (DCAB), which is determined by calculating the cumulative amount of sodium, potassium, calcium, and magnesium regularly consumed by the animal and subtracting the cumulative amount of chloride, sulfur, and phosphorus regularly consumed by the animal. See, Baker et al., Comparative Nutrition of Cats and Dogs, Ann. Rev. Nutr. 11:239-63 (1991).

In adjusting the balance of metabolizable cations to metabolizable anions consumed by the animal, it has generally been found that increasing the balance of metabolizable cations to metabolizable anions will result in firmer stool quality and reduced stool output. Conversely, decreasing the balance of metabolizable cations to metabolizable anions will result in looser stool and increased stool output.

In some embodiments wherein the animal is susceptible to or suffering from loose stool and/or frequent stool output, stool quality and stool frequency may be improved by increasing the balance of metabolizable cations to metabolizable anions to provide the animal with firmer stool and/or to reduce stool output. In such embodiments, the balance of cations to anions can be increased by increasing the cumulative amount of calcium, sodium, potassium, and magnesium regularly consumed by the animal relative to the cumulative amount of phosphorus, chloride, and sulfur regularly consumed by the animal. For example, the balance of metabolizable cations to metabolizable anions may be increased by increasing the animal's dietary intake of at least one composition comprising a cation excess of calcium, sodium, potassium, or magnesium. Likewise, the balance may be increased by decreasing the animal's dietary intake of at least one composition comprising an anion excess of chloride, phosphorus or sulfur.

In other embodiments wherein the animal is susceptible to or suffering from constipation, stool quality and/or stool frequency can be improved by decreasing the balance of metabolizable cations to metabolizable anions consumed by the animal to provide the animal with looser stool and/or increased stool output. In such embodiments, the balance of cations to anions can be decreased by decreasing the cumulative amount of calcium, sodium, potassium, and magnesium regularly consumed by the animal relative to the cumulative amount of phosphorus, chloride, and sulfur regularly consumed by the animal. For example, the balance of metabolizable cations to metabolizable anions can be decreased by decreasing the animal's dietary intake of at least one composition comprising a cation excess of calcium, sodium, potassium, or magnesium. Likewise, the balance may be decreased by increasing the animal's dietary intake of at least one composition comprising an anion excess of chloride, phosphorus or sulfur.

In some embodiments, the balance of metabolizable cations to metabolizable anions consumed by the animal can be adjusted by feeding the animal a composition having a stool quality altering amount of one or more metabolizable cations or metabolizable anions. Such compositions can include food compositions containing one or more ingredients suitable for consumption by an animal. In some embodiments, the food composition comprises a dry food (i.e., a food containing from about 3 to about 11% water). In other embodiments, the food composition comprises a semi-moist food (i.e., a food containing from about 25 to about 35% water). In some embodiments, the food composition comprises a moist food (i.e., a food containing from about 60 to more than about 87% water). In some embodiments, the food composition comprises a treat, snack, supplement, or partially or fully edible toy.

In some embodiments, the balance of metabolizable cations to metabolizable anions consumed by the animal can be adjusted in conjunction with the administration of one or more anti-diarrhea agents or anti-constipation agents. The term “anti-diarrhea agent” means any compound, composition, or drug useful for preventing or treating diarrhea. The term “anti-constipation agent” means any compound, composition, or drug useful for preventing or treating constipation.

In some embodiments, the balance of metabolizable cations to metabolizable anions consumed by the animal can be adjusted in conjunction with the administration of one or more compositions comprising a gastrointestinal tract improving agent selected from the group consisting of probiotics and prebiotics. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics.

Prebiotics are nondigestible food ingredients that beneficially affect host health by selectively stimulating the growth and/or activity of bacteria in the colon. The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly.

Probiotics enhance systemic cellular immune responses and may be useful as a dietary supplement to boost natural immunity in otherwise healthy adults. Probiotics include many types of bacteria but generally are selected from four genera of bacteria: Lactobacillius acidophillus, Bifidobacteria, Lactococcus, and Pediococcus. The amount of probiotics and prebiotics to be administered to the animal is determined by the skilled artisan based upon the type and nature of the probiotic and prebiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal.

In another aspect, the present invention provides kits suitable for altering stool quality and/or stool frequency comprising a stool quality altering amount or stool frequency altering amount of at least one metabolizable cation or metabolizable anion. In some embodiments, the kit further comprises one or more anti-diarrhea agents or anti-constipation agents, and/or one or more gastrointestinal tract improving agents selected from the group consisting of probiotics and prebiotics. In some embodiments, the kit further comprises instructions for at least one of (1) feeding the composition to an animal to adjust the balance of metabolizable cations to metabolizable anions consumed by the animal, or (2) administering an anti-diarrhea agent, an anti-constipation agent or a gastrointestinal tract improving agent in conjunction with the composition to alter an animal's stool quality or stool frequency.

In some embodiments, the kit comprises in separate containers in a single package or in separate containers in a virtual package, as appropriate for the kit component, a stool quality altering and/or stool frequency altering amount of at least one ingredient comprising a metabolizable cation or metabolizable anion and at least one of (1) an ingredient comprising a different metabolizable cation or metabolizable anion; (2) one or more ingredients for consumption by an animal; (3) one or more gastrointestinal tract improving agents selected from the group consisting of probiotics and prebiotics; (4) one or more anti-diarrhea agents; (5) one or more anti-constipation agents; (6) instructions for combining one or more relevant kit components to prepare a composition useful for altering stool quality and/or stool frequency; and (7) instructions for using one or more kit components to alter stool quality and/or stool frequency.

The term “single package” generally means that the components of a kit are physically associated in or with one or more containers and considered as a unit of manufacture, distribution, sale, or use. Containers include, but are not limited to, bags, boxes, bottles, shrink wrap packages, stapled or otherwise fixed components, or combinations thereof. A single package can be, for example, containers or individual food compositions physically associated such that they are considered a unit for manufacture, distribution, sale, or use. The term “virtual package” generally means that the components of a kit are associated by directions on one or more physical or virtual kit components instructing the user how to obtain components, e.g., in a bag containing one component and directions instructing the user to go to a website, contact a recorded message, view a visual message, or contact a caregiver to obtain instructions on how to use the kit. When the kit comprises a virtual package, the kit is limited to instructions in a virtual environment with one or more physical kit components.

In another aspect, the present invention provides a means for communicating information about or instructions for one or more of (1) using a balance of metabolizable cations to metabolizable anions consumed by an animal to alter stool quality and/or alter stool frequency, (2) admixing metabolizable cations and metabolizable anions with the other components described in the present invention, (3) administering metabolizable cations and metabolizable anions to an animal, alone or in combination with the other elements described in the present invention, and (4) using the kits of the present invention for altering stool quality and/or altering stool frequency, comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information or instructions include, for example, (1) information and instructions how to use a composition, method, or kit described in the present invention and (2) contact information for animal caregivers if they have a question about the invention and its uses.

In a further aspect, the present invention provides for a use of a composition that comprises a stool quality adjusting amount of at least one ingredient selected from the group consisting of metabolizable cations and metabolizable anions to prepare a medicament. In another, the invention provides for the use of such a composition to prepare a medicament for altering stool quality or altering stool frequency. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

### EXAMPLES

This invention can be further illustrated by the following example. However, it should be understood that the example is included merely for purposes of illustration and is not intended to limit the scope of the invention unless otherwise specifically indicated.

Example 1

Seventy dogs were randomly assigned to one of 7 groups containing 10 dogs per group. Each group received one of 7 foods formulated to contain a different balance of metabolizable cations to metabolizable anions. The balance of metabolizable cations to metabolizable anions was calculated as DCAB described above comprising the cumulative amount of sodium, potassium, calcium, and magnesium minus the cumulative amount of chloride, sulfur and phosphorus. The dogs were fed the foods for 7 days over which time the dog's stools were collected and scored. Each stool sample was scored on a scale of 1 to 5 with 1 being watery and loose and 5 being ideal.

Results showing the nutrient analysis of each food, the average stool score for each food, and the number of stools scored for each food are presented in Table 1.

TABLE 1
Analyzed nutrient composition of foods fed to dogs^{a}

| | Food | Food | Food | | | Food | Food |
|---|---|---|---|---|---|---|---|
| Item | 1 | 2 | 3 | Food 4 | Food 5 | 6 | 7 |
| Crude Protein (%) | 20.2 | 18.6 | 19.1 | 22.1 | 21.2 | 21.3 | 21.5 |
| Crude Fat (%) | 13.0 | 14.0 | 14.2 | 9.8 | 13.8 | 14.0 | 12.8 |
| Crude Fiber (%) | 2.9 | 3.0 | 3.2 | 1.4 | 4.1 | 4.7 | 3.7 |
| Sodium (%) | 0.39 | 0.29 | 0.30 | 0.30 | 0.43 | 0.43 | 0.39 |
| Potassium (%) | 0.87 | 0.64 | 0.71 | 0.63 | 1.29 | 1.55 | 1.52 |
| Chloride (%) | 0.97 | 0.77 | 0.74 | 0.72 | 0.83 | 0.79 | 0.76 |
| Sulfur (%) | 0.40 | 0.33 | 0.33 | 0.25 | 0.53 | 0.58 | 0.54 |
| Calcium (%) | 0.63 | 0.67 | 0.66 | 0.75 | 0.66 | 0.62 | 0.66 |
| Magnesium (%) | 0.06 | 0.05 | 0.05 | 0.16 | 0.09 | 0.11 | 0.11 |
| Phosphorus (%) | 0.58 | 0.55 | 0.50 | 0.75 | 0.58 | 0.63 | 0.62 |
| DCAB^{b} (meq) | −107 | −81 | −23 | +4 | +21 | +30 | +62 |
| Stool Score^{c} | 4.0 | 4.1 | 4.3 | 4.3 | 4.6 | 4.8 | 4.6 |
| Stool Count^{d} | 84 | 85 | 69 | 80 | 68 | 68 | 68 |

^{a}Dry Matter Basis
^{b}DCAB = (Sodium + Potassium + Calcium + Magnesium) − (Chloride + Sulfur + Phosphorous)
^{c}Stool scores were ranked from 1 to 5 with 1 being watery and loose and 5 being ideal
^{d}Number of stools scored over seven day period

The results show that the balance of metabolizable cations to metabolizable anions in the foods was directly related to dog stool quality and stool frequency. Dogs fed foods having a higher balance of metabolizable cations to metabolizable anions or DCAB demonstrated higher stool scores indicating improved stool quality and lower stool counts indicating a reduction in stool frequency.

In the specification, there have been disclosed typical preferred embodiments of the invention and, although specific terms are employed, they are used in a generic and descriptive sense only and not for purposes of limitation. Obviously many modifications and variations of the invention are possible in light of the above teachings. It is therefore to be understood that the invention may be practiced otherwise than as specifically described.

As used herein and in the appended claims, the singular forms “a”, “an”, and “the” include plural reference unless the context clearly indicates otherwise. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A method for improving stool quality comprising adjusting the balance of metabolizable cations to metabolizable anions consumed by an animal by an amount sufficient to improve stool quality.

2. The method of claim 1 wherein the metabolizable cations are selected from the group consisting of calcium, sodium, potassium, and magnesium.

3. The method of claim 1 wherein the metabolizable anions are selected from the group consisting of phosphorus, chloride, and sulfur.

4. The method of claim 1 wherein stool quality is improved by increasing the balance of metabolizable cations to metabolizable anions.

5. The method of claim 4 wherein the balance is increased by increasing the cumulative amount of calcium sodium, potassium, and magnesium regularly consumed by the animal relative to the cumulative amount of phosphorus, chloride, and sulfur regularly consumed by the animal.

6. The method of claim 4 wherein the balance is increased by increasing the animal's dietary intake of at least one composition comprising a cation excess of calcium, sodium, potassium, or magnesium.

7. The method of claim 4 wherein the balance is increased by decreasing the animal's dietary intake of at least one composition comprising an anion excess of phosphorus, chloride, or sulfur.

8. The method of claim 1 further comprising administering to the animal at least one compound selected from the group consisting of one or more gastrointestinal tract improving agents and one or more anti-diarrhea agents.

9. The method of claim 1 wherein stool quality is improved by decreasing the balance of metabolizable cations to metabolizable anions.

10. The method of claim 9 wherein the balance is decreased by decreasing the cumulative amount of calcium, sodium, potassium, and magnesium regularly consumed by the animal relative to the cumulative amount of phosphorus, chloride, and sulfur regularly consumed by the animal.

11. The method of claim 9 wherein the balance is decreased by decreasing the animal's dietary intake of at least one composition comprising a cation excess of calcium, sodium, potassium, or magnesium.

12. The method of claim 9 wherein the balance is decreased by increasing the animal's dietary intake of at least one composition comprising an anion excess of phosphorus, chloride, or sulfur.

13. The method of claim 9 further comprising administering to the animal at least one compound selected from the group consisting of one or more gastrointestinal tract improving agents and one or more anti-constipation agents.

14. A method for altering stool frequency comprising adjusting the balance of metabolizable cations to metabolizable anions consumed by an animal by an amount sufficient to alter stool frequency.

15. The method of claim 14 wherein the metabolizable cation is selected from the group consisting of calcium, sodium, potassium, and magnesium.

16. The method of claim 14 wherein the metabolizable anion is selected from the group consisting of phosphorus, chloride, and sulfur.

17. The method of claim 14 wherein stool frequency is reduced by increasing the balance of metabolizable cations to metabolizable anions.

18. The method of claim 14 wherein stool frequency is increased by decreasing the balance of metabolizable cations to metabolizable anions.

19. The method of claim 14 further comprising administering to the animal at least one compound selected from the group consisting of gastrointestinal tract improving agents, anti-diarrhea agents, and anti-constipation agents.

20. A kit suitable for altering stool quality and/or stool frequency comprising in separate containers in a single package or in separate containers in a virtual package, as appropriate for the kit component, a stool quality altering and/or stool frequency altering amount of at least one metabolizable cation or metabolizable anion and at least one of (1) an ingredient comprising a different metabolizable cation or metabolizable anion; (2) one or more ingredients for consumption by an animal; (3) one or more gastrointestinal tract improving agents selected from the group consisting of probiotics and prebiotics; (4) one or more anti-diarrhea agents; (5) one or more anti-constipation agents; (6) instructions for combining one or more relevant kit components to prepare a composition useful for altering stool quality and/or stool frequency; and (7) instructions for using one or more kit components to alter stool quality and/or stool frequency.

21. A means for communicating information about or instructions for one or more of (1) using a balance of metabolizable cations to metabolizable anions consumed by an animal to alter stool quality and/or alter stool frequency, (2) admixing metabolizable cations and metabolizable anions with the other components of the present invention, (3) administering metabolizable cations and metabolizable anions to an animal, alone or in combination with the other elements of the present invention, and (4) using the kits of the present invention for altering stool quality and/or altering stool frequency, the means comprising a document, digital storage media, audio presentation, or visual display containing the information or instructions.

22. The means of claim 21 selected from the group consisting of a displayed website, brochure, product label package insert, advertisement, displayed web site, and visual display.

23. A use of a composition that comprises a stool quality or stool frequency altering amount of at least one ingredient selected from the group consisting of metabolizable cations and metabolizable anions to prepare a medicament for altering stool quality or altering stool frequency.
